(19) United States
(12) Patent Application Publication
(10) Pub. No.: US 2012/0135532 A1
(43) Pub. Date: May 31, 2012

(54) Sensor Comprising Resrufin Levulinate Having Sulfite Ion Selectivity and Method for Monitoring Sulfite Ion Using the Same

(75) Inventors: Suk-Kyu CHANG, Gyeonggi-do (KR); Myung Gil CHOI, Gyeonggi-do (KR); Suyoung EOR, Seoul (KR); Jiyoung HWANG, Seoul (KR)

(73) Assignee: Chung-Ang University Industry-Academiy Cooperation Foundation, Seoul (KR)

(21) Appl. No.: 13/282,214

(22) Filed: Oct. 26, 2011

(30) Foreign Application Priority Data
Nov. 30, 2010 (KR) 10-2010-0120831

(51) Int. Cl. G01N 33/00; C07D 265/38

(52) U.S. Cl. 436/119; 544/102

(57) ABSTRACT

The present invention relates to a sensor comprising a resorufin compound having sulfite ion selectivity and a method for detecting sulfite ions using the same. More specifically, the resorufin compound may have outstandingly increased fluorescence intensity by a deprotection reaction that a levulinyl group is cleaved with a sulfite ion to be used as a selective fluorescence sensor of turn-on type, and also represent a chromogenic change to detect sulfite ions by naked eye.

### TECHNICAL FIELD

The present invention relates to a sensor comprising resorufin levulinate having sulfite ion selectivity and a method for monitoring sulfite ion using the same.

### BACKGROUND ART

Sulfites are widely used as a preservative in foods and beverages. To develop analysis for measuring a concentration of sulfites is important for consumer safety. Sulfites are known to be related to allergy reactions and food intolerance. The most frequent conditions caused by sulfites are not only bowel diseases, but also asthma and allergy type such as difficulty breathing, wheeze, or hives. Sulfites have a potential toxicity and are severely restricted to have an acceptable daily intake of 0.7 mg per kg of body weight.

Therefore, to develop any convenient analysis of sulfites is important in view of food safety and quality control. Sulfites in foods and beverages are measured by general methods such as a titrimetry, chromatography, an electrochemisty, a capillary electrophoresis and a flow injection analysis. However, the convenient methods for analyzing sulfites require pre-treatment of the troublesome sample and reagent production, time-consuming and require complicated instruments unsuitable in routine tests. For this reason, more convenient tools such as optical sensors and chromoreactants have been a lot of research interest.

In manufacturing many sophisticated signaling systems, the only signaling by chemodosimeter or selective chemical modification of chemical probes has been used. Representative examples of such an approach are Cu²⁺ signaling by hydrolysis of rhodamine hydrazide and peroxide hydrogen visualizing by boronate deprotection of fluorescein and resorufin. In addition, there are as a successfully designed system probes for signaling fluoride, cyanide, sulfide, phosphate, Cu²⁺ and Hg²⁺ ions.

Also, a levulinyl group is frequently used as a protecting group of a hydroxyl group in nucleotides, peptides and sugars. Ono, et al. have reported that revulinates protecting phenyl moieties can be easily and selectively deprotected by the sulfite ions under mild neutral conditions (Chem. Lett., 1988, p 585). Based on this fact, the present inventors manufactured novel sulfite ion selective probes which may generate chromogenic and fluorogenic signaling, being capable of detecting by naked eye, to complete the present invention.

### SUMMARY OF INVENTION

### Technical Problem

The present invention is intended to provide novel chromogenic and fluorescence signaling systems of sulfite ions, based on selective deprotection of resorufin compounds.

### Solution of Problem

To accomplish the above object, the present invention provides a compound represented by the following chemical formula 1.

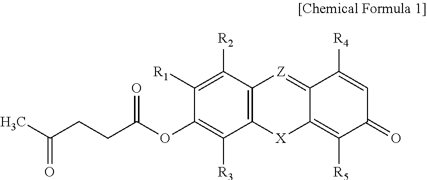

wherein,

R₁ to R₅ represent each independently hydrogen, halogen, carboxyl, cyano, nitro, alkoxy with 1 to 4 carbon atoms, or alkyl with 1 to 4 carbon atoms,

X is an oxygen atom or a sulfur atom, and

Z represents a nitrogen atom.

The present invention provides a sensor for detecting sulfite ions comprising a compound represented by the following chemical formula 1.

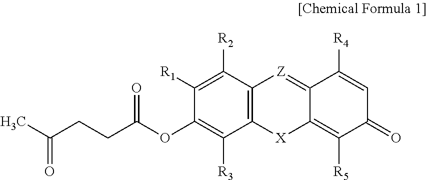

wherein,

R₁ to R₅ represent each independently hydrogen, halogen, carboxyl, cyano, nitro, alkoxy with 1 to 4 carbon atoms, or alkyl with 1 to 4 carbon atoms,

X is an oxygen atom or a sulfur atom, and

Z represents a nitrogen atom.

The present invention also provides a composition for detecting sulfite ions comprising a compound represented by said chemical formula 1,

The present invention also provides a method for detecting sulfite ion comprising a step of being subjected to reaction of a compound represented by said chemical formula 1 with a sample containing sulfite ions.

### Advantageous Effects of Invention

The present resorufin compounds may be used as a turn-on type selective fluorescence sensor for sulfite ions, since they release levulinyl groups by sulfite ions through a selective deprotection reaction to increase outstandingly fluorescence intensity.

In addition, when said deprotection reaction occurs, color in an aqueous solution may be changed from yellow to pink to detect by naked eye.

### BRIEF DESCRIPTION OF DRAWINGS

The file of this patent contains at least one drawing executed in color. Copies of this patent with color drawings will be provided by the Office upon request and payment of the necessary fee.

FIG. 1 is a UV-vis spectrum depicting a result of reacting resorufin levulinate (1.0×10⁻⁵M) of the present invention and general anions ([Aⁿ⁻]=1.0×10⁻³M in an aqueous solution, pH 7.0, of 10 mM acetonitrile (H₂O—CH₃CN=98:2, v/v) buffered with a HEPES buffer solution.

FIG. 2 is a graph representing absorbance ratios (A₅₇₁/A₃₅₉) after reacting resorufin levulinate (1.0×10⁻⁵M) of the present invention and anions ([Aⁿ⁻]=1.0×10⁻³M) in an aqueous solution, pH 7.0, of 10 mM acetonitrile (H₂O—CH₃CN=98:2, v/v) buffered with a HEPES buffer solution.

FIG. 3 is a fluorescence spectrum (λₑₓ=487 nm) depicting a result of reacting resorufin levulinate (5.0×10⁻⁶M) of the present invention and general physiologically and environmentally related anions ([Aⁿ⁻]=5.0×10⁻⁴M) in an aqueous solution, pH 7.0, of 10 mM acetonitrile (H₂O—CH₃CN=98:2, v/v) buffered with a HEPES buffer solution.

FIG. 4 is a graph (λₑₓ=487 nm) representing change in fluorescence intensity ratios (I/I₀) at 588 nm after reacting resorufin levulinate (5.0×10⁻⁶M) of the present invention and general physiologically and environmentally related anions ([Aⁿ⁻]=5.0×10⁻⁴M) in an aqueous solution, pH 7.0, of 10 mM acetonitrile (H₂O—CH₃CN=98:2, v/v) buffered with a HEPES buffer solution.

FIG. 5 represents partial spectrums of resorufin levulinate (5.0×10⁻³M), resorufin levulinate+sulfite ion (1.0×10⁻¹M), and resorufin (5.0×10⁻³M)+sulfite ion in a deuterated aqueous solution of acetonitrile (D₂O—CD₃CN, 50:50, v/v).

FIG. 6 represents a UV-vis titration of resorufin levulinate (1.0×10⁻⁵M) of the present invention and sulfite ion in an aqueous solution, pH 7.0, of 10 mM acetonitrile (H₂O—CH₃CN=98:2, v/v) buffered with a HEPES buffer solution.

FIG. 7 represents results of competitive experiments for a resorufin levulinate (1.0×10⁻⁵M) of the present invention−sulfite ion (2.0×10⁻⁴M) system in presence of coexisting anions ([Aⁿ⁻]=1.0×10⁻³M) in an aqueous solution, pH 7.0, of 10 mM acetonitrile (H₂O—CH₃CN=98:2, v/v) buffered with a HEPES buffer solution.

FIG. 8 represents signaling of sulfite ions (2.0×10⁻⁴M) by resorufin levulinate (1.0×10⁻⁵M) of the present invention in presence of coherent anions (1.0×10⁻³M) in an aqueous solution, pH 7.0, of 10 μM acetonitrile (H₂O—CH₃CN=98:2, v/v) buffered with a HEPES buffer solution, wherein a) is a result of a UV-vis spectrum, and b) is an absorption (A_{1+anion+sulfite}/A_{1+sulfite}) calculated at 571 nm.

FIG. 9 represents minutely signaling behavior of resorufin levulinate (1.0×10⁻⁵M) of the present invention and sulfite ion (1.0×10⁻³M) in an aqueous solution, pH 7.0, of 10 mM acetonitrile (H₂O—CH₃CN=98:2, v/v) buffered with a HEPES buffer solution.

### DESCRIPTION OF EMBODIMENTS

The constitution of the present invention is explained in detail below.

The present invention relates to a compound represented by the following chemical formula 1:

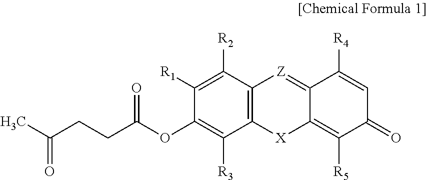

wherein,

R₁ to R₅ represent each independently hydrogen, halogen, carboxyl, cyano, nitro, alkoxy with 1 to 4 carbon atoms, or alkyl with 1 to 4 carbon atoms,

X is an oxygen atom or a sulfur atom, and

Z represents a nitrogen atom.

Terms used in substituent definitions of the present compounds are as follows.

“Halogen” is —F, —Cl, —Br or —I.

“Alkyl” indicates alkyl with 1 to 4 carbon atoms, for example saturated straight, branched or cyclic hydrocarbons with 1 to 4 carbon atoms, unless otherwise described. An example of C₁₋₄ alkyl groups includes methyl, ethyl, propyl, butyl, isobutyl, sec-butyl, or tert-butyl, and the like, but is not limited thereto.

“Alkoxy” indicates alkoxy with 1 to 4 carbon atoms, for example, one that an alkyl group with 1 to 4 carbon atoms is linked with an oxygen atom, unless otherwise described. An example of C₁₋₄ alkoxy groups includes methoxy, ethoxy, propoxy, and butoxy, but is not limited thereto.

A specific example of said compounds of chemical formula 1 may be a compound wherein R₁ to R₅ are hydrogen, and X represents an oxygen atom.

Said resorufin compounds of chemical formula 1 may be used as a fluorescence sensor for selectively detecting sulfite ions by increasing fluorescence intensity through deprotection reaction that levulinyl groups are released by sulfite ions.

In addition, it is characterized in that when said deprotection reaction occurs under an aqueous solution, color of the aqueous solution represents color change from yellow to pink, so that sulfite ions may be identified by naked eye.

The present invention also relates to a sensor for detecting sulfite ions comprising a compound represented by the following chemical formula 1:

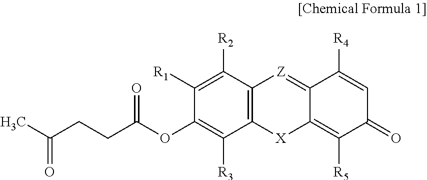

wherein,

R₁ to R₅ represent each independently hydrogen, halogen, carboxyl, cyano, nitro, alkoxy with 1 to 4 carbon atoms, or alkyl with 1 to 4 carbon atoms,

X is an oxygen atom or a sulfur atom, and

Z represents a nitrogen atom.

A specific example of said compounds of chemical formula 1 may be a compound wherein R₁ to R₅ are hydrogen, and X represents an oxygen atom.

Said resorufin compounds of chemical formula 1 may be used as a fluorescence sensor for selectively detecting sulfite ions by increasing fluorescence intensity through deprotection reaction that levulinyl groups are released by sulfite ions. According to mechanism of the following reaction scheme 1, chromogenic and fluorogenic signaling is due to selective deprotection of resorufin levulinate by the sulfite ions. In cleavage of levulinate, an initial attack of a sulfite ion to the carbonyl carbon at the 4-position of levulinate forms a tetrahedral intermediate and then intramolecular cyclization leads cleavage of an ester functional group. Therefore, the resulting resorufin represents its own characteristic chromogenic and fluorogenic signaling behavior. Accordingly, the resorufin compounds of chemical formula 1 represent strong fluorescence, with being converted to resorufin through deprotection reaction that levulinyl groups are released by sulfite ions and colorimetric and fluorogenic signaling characteristic of turn on type which represents color change from yellow to pink:

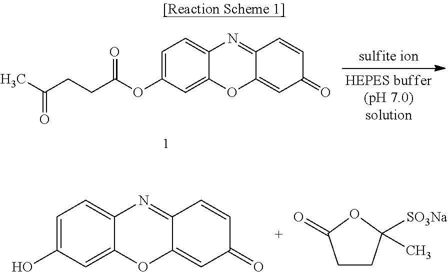

The resorufin compounds of chemical formula 1 according to the present invention represent concentration-dependently selective fluorescence increase at a wavelength of 588 nm as sulfite ions are added in an aqueous solution, and thus said resorufin compounds may be used as a fluorescence probe of turn-on type to detect the sulfite ion.

According to one embodiment of the present invention, the resorufin compounds of chemical formula 1 of the present invention do not represent fluorescence change for any anion such as F⁻, Cl⁻, Br⁻, I⁻, SO₄²⁻, HPO₃²⁻, NO₃⁻, N₃⁻, AcO⁻, ClO₄⁻, or HCO₃⁻, and the like, but concentration-dependently a wide change of fluorescence, if they are reacted with sulfite ions.

According to one embodiment of the present invention, the resorufin compounds of chemical formula 1 of the present invention are selectively reacted with sulfite ions even in presence of an alkali metal ion (Li⁺, Na⁺, K⁺), an alkali earth metal ion (Mg²⁺, Ca²⁺), or a transition metal ion (Fe³⁺, Ni²⁺, Zn²⁺, Ba²⁺, Co²⁺, Cd²⁺), and the like, to represent a wide change of fluorescence.

In addition, said compounds of chemical formula 1 represent a suitable UV-vis absorption at 359 and 456 nm, but concentration-dependently a widely increased absorbance at 571 nm through a selective reaction with sulfite ions, so that signals of sulfite ions may be also measured by measuring this absorbance.

Furthermore, detection of sulfite ions may be also measured by naked eye through colorimetric change.

According to one embodiment, the resorufin compounds according to the present invention represent colorimetric change from yellow to pink, as a concentration of sulfite ions which are reacted with them in an aqueous solution increases.

The sensor for detecting sulfite ions of the present invention may be provided as a usual kit which may be mixed with a sample solution to detect sulfite ions to identify presence of the sulfite ion and a concentration thereof.

The present invention relates to a composition for detecting sulfite ions comprising a compound represented by the above chemical formula 1.

The composition for detecting sulfite ions of the present invention may comprise a buffer solution in addition to the compound represented by chemical formula 1. The buffer solution is not particularly limited to any kind and concentration, which will be suitably changed depending on an appropriate use of said composition for detecting sulfite ions. More specifically, it may be a buffer solution having pH 7 to 10. Most specifically, an aqueous solution of acetonitrile having pH 7 to 10 buffered with a HEPES buffer solution may be used.

The present invention also relates to a method for detecting sulfite ions comprising a step of reacting said compound represented by chemical formula 1 with a sample containing sulfite ions.

The resorufin compound of chemical formula 1 according to the present invention is a turn-on type sensor to be capable of detecting sulfite ions in a state of an aqueous solution, the type of which is made up by a fact that fluorescence intensity is amplified when it detects sulfite ions in a state of showing weak fluorescence, and may have a fast reaction rate to promptly detect sulfite ions.

According to one embodiment of the present invention, said reaction is completed within about 15 minutes, so that it is possible to promptly detect sulfite ions.

In addition, detection of sulfite ions by said resorufin compound of chemical formula 1 may be carried out under an aqueous solution or a mixed aqueous solution containing an organic solvent such as methanol, acetonitrile, tetrahydrofuran, dimethylsulfoxide, or dioxane.

Furthermore, since said detection reaction does not occur in an acidic condition, it is carried out in an aqueous solution at, preferably, pH 7 to 10. Most specifically, an aqueous solution of acetonitrile having pH 7 to 10 buffered with a HEPES buffer solution may be used, but is not particularly limited thereto.

Furthermore, detection of sulfite ions by said resorufin compound of chemical formula 1 is to measure change of fluorescence intensity, and the fluorescence intensity may be measured, as it increases in a sulfite ion concentration dependent manner.

For detection of sulfite ions, absorbance may be also measured, as it increases widely in a sulfite ion dependent manner at 571 nm.

According to one embodiment of the present invention, the resorufin compound represents absorbance ratio 160 times or higher after reacting with sulfite ions.

In addition, detection of said sulfite ions may be measured via colorimetric change of the aqueous solution.

According to one embodiment of the present invention, it may be observed by naked eye to change color of the resorufin compound in the aqueous solution from yellow to pink after reacting with sulfite ions.

Hereinafter, the present invention is explained in more detail via examples according to the present invention, but the scope of the present invention is not restricted by the following examples.

### Example 1

### Preparation of Resorufin Compounds

A method of preparing resorufin compounds to detect sulfite ions was briefly depicted in the following reaction scheme 2.

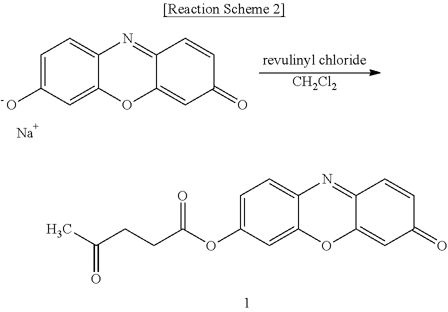

Resorufin sodium salt and levulinic acid were purchased from Aldrich Chemical Co. As all the solvents, products having a level of spectrometric grade manufactured by Aldrich Chemical Co. were used. ¹H NMR (600 MHz) and ¹³C NMR (150 MHz) spectrums were obtained from Varian VNS spectrometer, and referenced to signals of remaining solvents. UV-Vis spectrums were recorded using Jasco V-550 spectrophotometer fixed with Peltier temperature controllers. Fluorescence spectrums were measured from Aminco-Bowman Series 2 spectrophotometer. Mass spectrums were obtained from Micromass Autospec Mass Spectrometer.

To prepare resorufin compounds, oxalyl chloride (0.82 mL, 8.6 mmol) and DMF (15 μl) were added to a suspension of levulinic acid (500 mg, 4.3 mmol) dissolved in dichloromethane (50 mL). The reaction mixture was stirred at room temperature for 4 hours, and then volatile materials were distilled under reduced pressure and the residue was dried via vacuum pumping. The residue was dissolved in a small amount of dry dichloromethane. The above solution was slowly added to the dispersed dichloromethane solution (50 mL) containing resorufin sodium salt (300 mg, 1.3 mmol) and triethylamine (0.54 mL, 3.9 mmol). After stirring the mixture for 12 hours, the reaction mixture was filtered off and the resulting solution was treated with water. The organic phase was separated, washed with 1M sodium bicarbonate solution and water, and then distilled to obtain a residue in a solid phase. The final product was crystallized from ethyl acetate and purified. The yield was 75%.

¹H NMR (600 MHz, CDCl₃) d7.77 (d, J=8.6 Hz, 1H), 7.41 (d, J=9.8 Hz, 1H), 7.13 (s, 1H), 7.12 (d, J=8.6 Hz, 1H), 6.84 (d, J=9.8 Hz, 1H), 6.30 (s, 1H), 2.89 (m, 2H), 2.83 (m, 2H), 2.23 (s, 3H);

¹³C NMR (150 MHz, CDCl₃) d206.1, 186.3, 170.7, 153.5, 149.3, 148.2, 144.3, 135.1, 134.8, 131.2, 131.1, 119.3, 109.7, 107.2, 37.8, 29.8, 28.2;

HRMS (DPI); m/z calcd for C₁₇H₁₃NO₅ [M]⁺: 311.0794, found 311.0786.

As in the above reaction scheme 2, a derivate of resorufin levulinate was prepared in high yield via reaction with levulinyl chloride (75%).

### Experimental Example 1

### Chromogenic and Fluorescence Behavior Measurement of Resorufin Compounds

Chromogenic signaling behavior of resorufin levulinate in an aqueous solution was investigated using the minimum amount of acetonitrile (H₂O:CH₃CN=98:2, v/v) as a solubilizer in 10 mM HEPES buffer solution (pH 7.0).

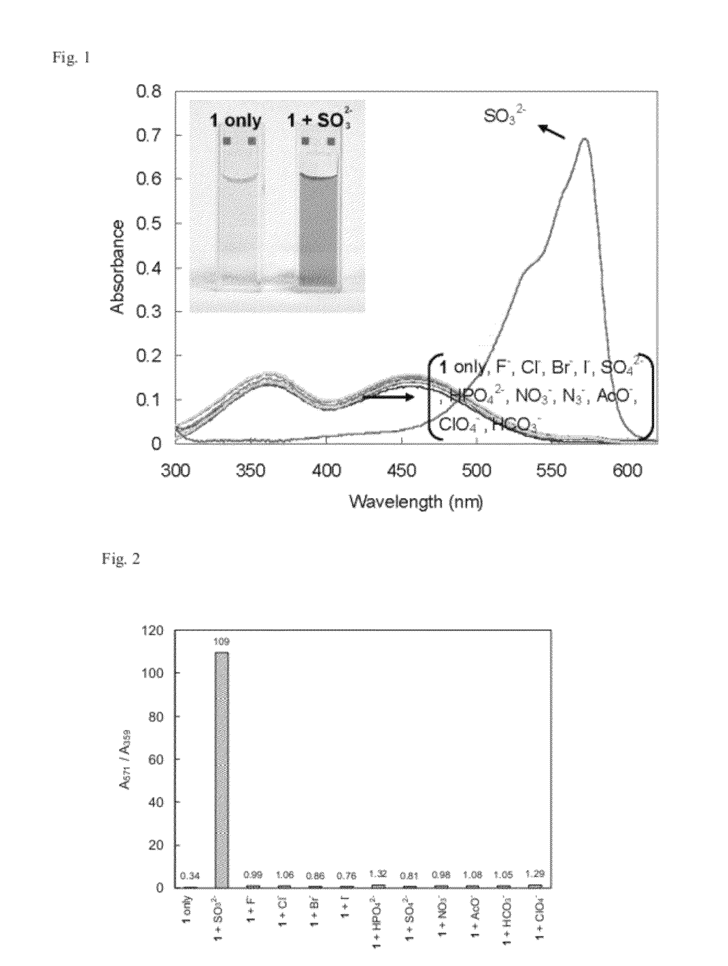

Resorufin levulinate represented a suitable UV-vis absorption at 359 and 456 nm. When it was reacted with 100 equivalents of a sulfite (Na₂SO₃), it represented strong concentrated absorption bands at 571 nm (FIG. 1). By representing dominant pink being a characteristic of resorufin at the same time, the chromogenic detection of sulfite ions was possible.

As reported in other signaling system based on resorufin by deprotecting into resorufin, absorption profile had high change. In case of sulfite ions, absorption ratio (A₅₇₁/A₃₅₉) at two characteristic wavelengths of 571 and 359 nm increased 160 times or more.

Other general anions had relatively no reaction and the absorption ratio (A₅₇₁/A₃₅₉) was varied in a certain range between 0.76 (in case of I⁻) and 1.81 (in case of ClO₄⁻) (FIG. 2).

Next, fluorescence signaling behavior of resorufin levulinate for sulfite ions was measured.

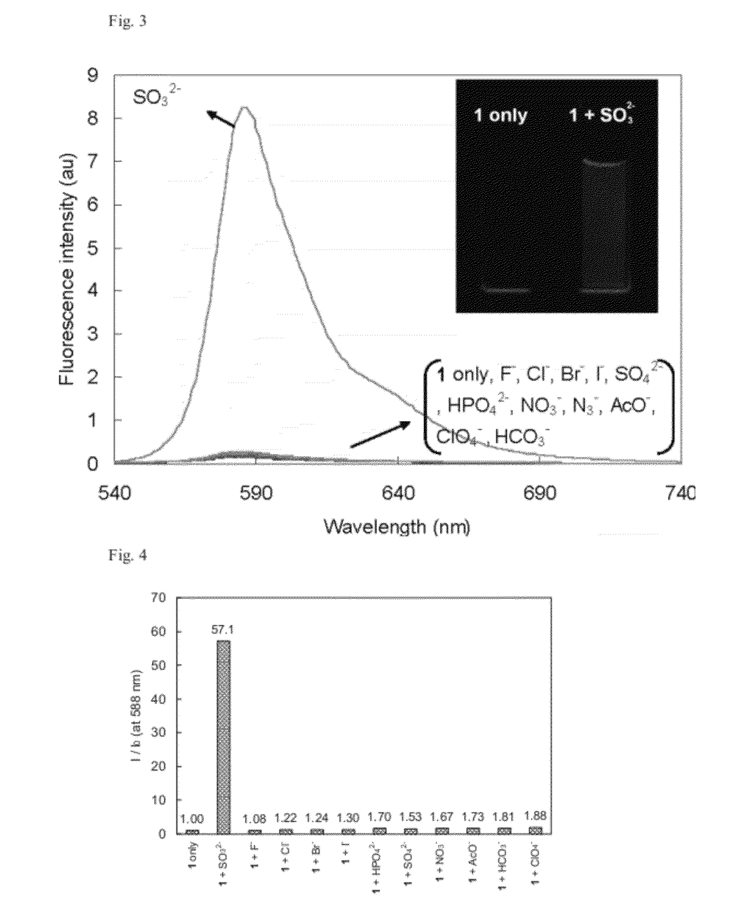

Resorufin levulinate represented weak emission at 584 nm. However, when it was treated with 100 equivalents of sulfite ions, strong emission was represented at 588 nm (FIG. 3). Fluorescence enhancement factor (I/I₀) observed at 588 nm was very high (57 times), and the aqueous solution showed dramatic color change from black to dark pink, when it was lighted with a UV lamp. Other general anions had relatively no reaction, I/I₀ at 588 nm was varied in a certain range between 1.08 (F⁻) and 1.88 (ClO₄⁻) (FIG. 4).

Chromogenic and fluorescence signaling is because of selective deprotection of resorufin levulinate by sulfite ions (Reaction Scheme 1). In cleavage of levulinate, an initial attack of a sulfite ion to the carbonyl carbon at the 4-position of levulinate formed a tetrahedral intermediate and then intramolecular cyclization leaded cleavage of an ester functional group. Therefore, the resulting resorufin represented its own characteristic chromogenic and fluorogenic signaling behavior.

The proposed modification by sulfite ions was proved by measuring NMR, UV-vis and fluorescence.

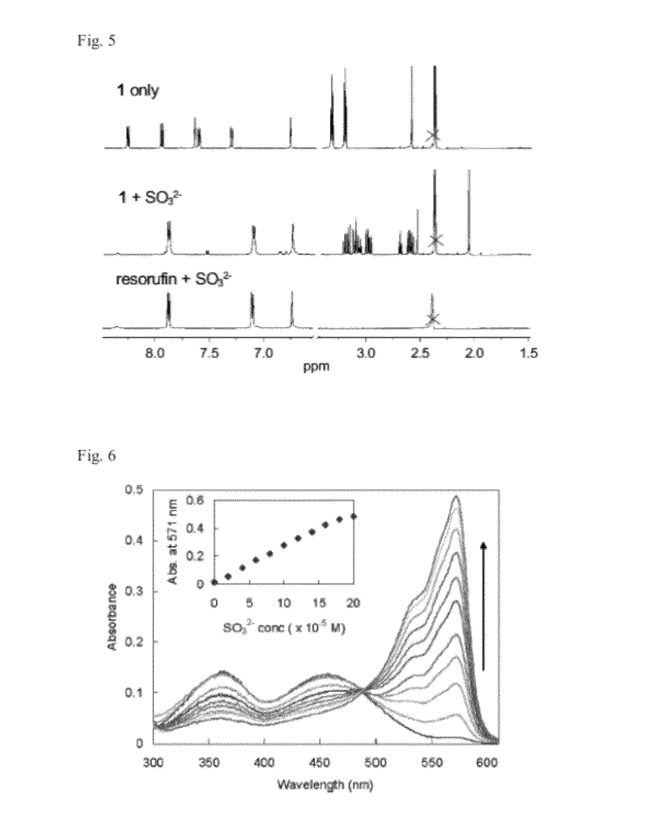

¹H NMR spectrums of resorufin levulinate under 20 equivalents of sulfite ions were virtually the same as those of resorufin having additional remaining peaks of sulfonate by-product near 2.1, 2.6-2.7 and 3.0-3.2 ppm (FIG. 5).

UV-vis and fluorescence spectrums of resorufin levulinate-sulfite ion system obtained by interaction of 100 equivalents of a sulfite and resorufin levulinate (1.0×10⁻⁵M) were virtually the same as those of resorufin.

Quantitative analysis behavior of resorufin levulinate for analyzing sulfite ions was investigated via UV-vis titration.

As concentration of sulfite ions increased, absorbance at 571 nm continuously increased for about 20 equivalents of sulfite ions (FIG. 6). As a result of titration, the limit of detecting resorufin levulinate for analyzing sulfite ions was evaluated to have 4.9×10⁻⁵M (4.0 ppm) in a 2% aqueous solution of acetonitrile.

### Experimental Example 2

### Selective Detection of Sulfite Ions in Presence of Other Metal Ions

Virtually applied possibility of sulfite ion signaling by resorufin levulinate was identified via competition experiments with generally encountered anions as well as metal ions.

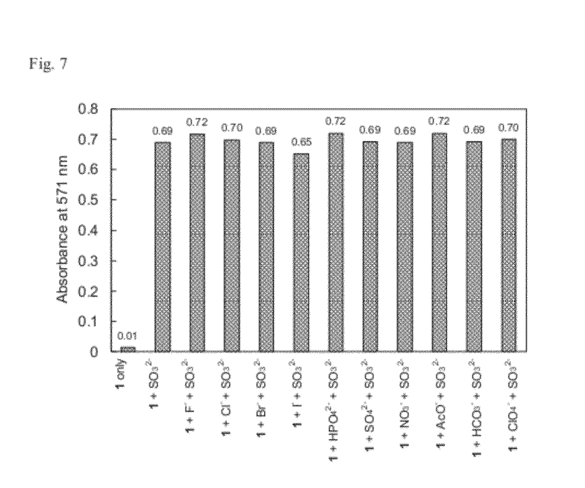

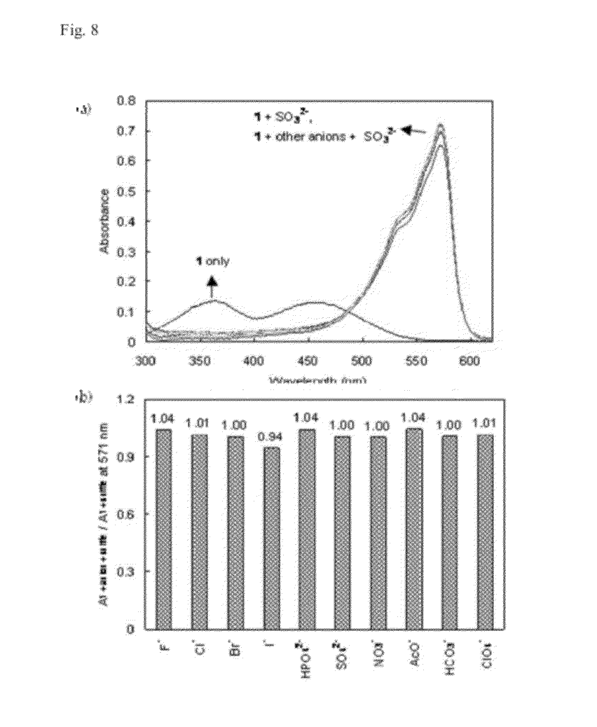

Signaling of resorufin levulinate by sulfite ions was not induced by 5 equivalents of the coexisting representative anions (FIGS. 7 and 8), and interference by other anions appeared, as absorption ratio (A_{1+Sulfite+Anion}/A_{1+Sulfite}) at 571 nm was varied in a certain range between 0.94 for iodide and 1.04 for fluoride.

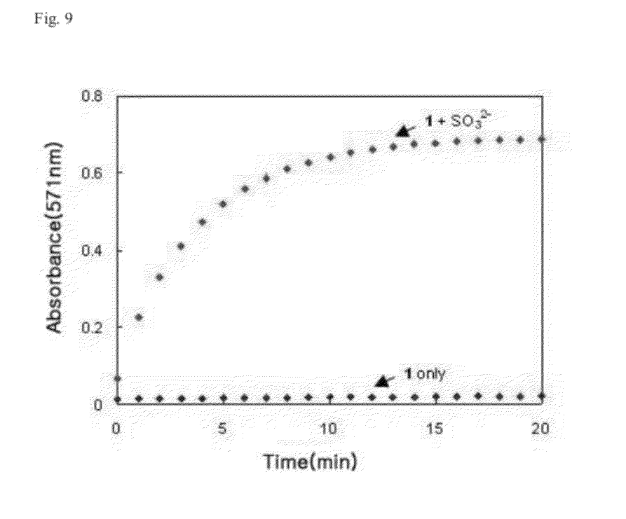

Signaling of sulfite ions by resorufin levulinate according to the present invention was relatively fast, which was completed within 15 minutes after preparing the sample (FIG. 9).

This means that the designed resorufin levulinate may be utilized as selective and efficient signaling probes for sulfite ions in an aqueous environment.

In summary, novel selective probes for sulfite ions were designed using sulfite ion-selective deprotection of levulinate. Using representative signaling moieties of resorufin, sulfite ion-selective chromogenic and fluorescence signaling systems were clearly embodied. The developed systems may be utilized, in generally chemical analyzing materials, as convenient and practical signaling apparatuses for optical measurement of sulfite ions in an aqueous environment.

1. A compound represented by the following chemical formula 1: 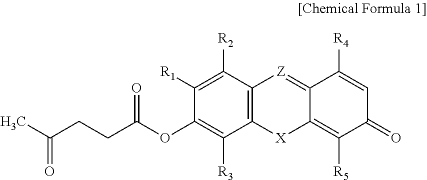
wherein,
R₁ to R₅ represent each independently hydrogen, halogen, carboxyl, cyano, nitro, alkoxy with 1 to 4 carbon atoms, or alkyl with 1 to 4 carbon atoms,
X is an oxygen atom or a sulfur atom, and
Z represents a nitrogen atom.

2. The compound according to claim 1, wherein
R₁ to R₅ are hydrogen, and
X represents an oxygen atom.

3. A sensor for detecting sulfite ions comprising a compound represented by the following chemical formula 1: 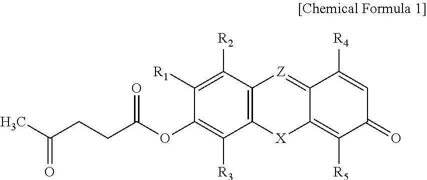
wherein,
R₁ to R₅ represent each independently hydrogen, halogen, carboxyl, cyano, nitro, alkoxy with 1 to 4 carbon atoms, or alkyl with 1 to 4 carbon atoms,
X is an oxygen atom or a sulfur atom, and
Z represents a nitrogen atom.

4. The sensor for detecting sulfite ions according to claim 3, wherein
R₁ to R₅ are hydrogen, and
X represents an oxygen atom.

5. A composition for detecting sulfite ions comprising a compound represented by the following chemical formula 1: 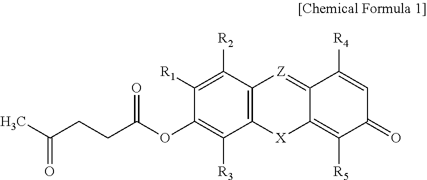
wherein,
R₁ to R₅ represent each independently hydrogen, halogen, carboxyl, cyano, nitro, alkoxy with 1 to 4 carbon atoms, or alkyl with 1 to 4 carbon atoms,
X is an oxygen atom or a sulfur atom, and
Z represents a nitrogen atom.

6. The composition for detecting sulfite ions according to claim 5, wherein R₁ to R₅ are hydrogen, and X represents an oxygen atom.

7. A method for detecting sulfite ions comprising a step of being subjected to reaction of a compound represented by the following chemical formula 1 with a sample containing sulfite ions: 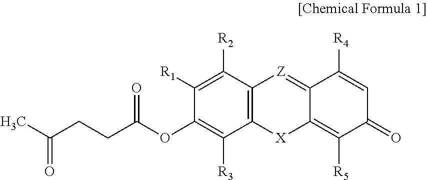
wherein,
R₁ to R₅ represent each independently hydrogen, halogen, carboxyl, cyano, nitro, alkoxy with 1 to 4 carbon atoms, or alkyl with 1 to 4 carbon atoms,
X is an oxygen atom or a sulfur atom, and
Z represents a nitrogen atom.

8. The method for detecting sulfite ions according to claim 7, wherein R₁ to R₅ are hydrogen, and X represents an oxygen atom.

9. The method for detecting sulfite ions according to claim 7, wherein the reaction is carried out under an aqueous solution having pH 7 to 10.

10. The method for detecting sulfite ions according to claim 9, wherein the aqueous solution is an aqueous solution of acetonitrile buffered with a HEPES buffer solution.

11. The method for detecting sulfite ions according to claim 7, wherein for detecting sulfite ions, increase of fluorescence intensity is measured.

12. The method for detecting sulfite ions according to claim 7, wherein for detecting sulfite ions, absorbance at 571 nm is measured.

13. The method for detecting sulfite ions according to claim 7, wherein for detecting sulfite ions, change of color in an aqueous solution from yellow to pink is measured.
